(19) United States
(12) Patent Application Publication
(10) Pub. No.: US 2014/0039598 A1
(43) Pub. Date: Feb. 6, 2014

(54) BLOOD LOSS CONTROL SYSTEM

(71) Applicants: Gregory C. Sampognaro, Monroe, LA (US); Stephen R. Ramee, New Orleans, CA (US); Gwendolyn A. Watanabe, Los Altos Hills, CA (US); Jeffrey Krolik, Campbell, CA (US)

(72) Inventors: Gregory C. Sampognaro, Monroe, LA (US); Stephen R. Ramee, New Orleans, CA (US); Gwendolyn A. Watanabe, Los Altos Hills, CA (US); Jeffrey Krolik, Campbell, CA (US)

(21) Appl. No.: 13/954,806

(22) Filed: Jul. 30, 2013

Related U.S. Application Data
(60) Provisional application No. 61/741,950, filed on Jul. 30, 2012.

(51) Int. Cl. A61F 2/958

(52) U.S. Cl. CPC A61F 2/958; A61F 2002/9528
USPC 623/1.11

(57) ABSTRACT

Generally described here are systems and methods for controlling bleeding. The systems generally comprise a blood control catheter having an expandable member, an intra-vessel support delivery device, and an intra-vessel support releasably attached to the intra-vessel support delivery device. The system may also include a guidewire. In some variations, the guidewire may be moveable between a tracking configuration and a delivery configuration.

### CROSS-REFERENCE TO RELATED APPLICATIONS

This application claims priority to U.S. Provisional Patent Application Ser. No. 61/741,950, filed Jul. 30, 2012 and titled “Blood loss control system”, the disclosure of which is incorporated by reference herein in its entirety.

### FIELD

The devices and methods described herein are generally directed to controlling bleeding.

### BACKGROUND

Endovascular procedures are an increasingly common alternative to open surgical procedures. Conducted from the interior of a blood vessel, endovascular procedures can be performed under local anesthesia with no (or partial) cardiac bypass, and require a shorter hospitalization than open surgical procedures. Prior to or during an endovascular procedure, access to the vasculature is obtained via one or more arteriotomies or other openings formed in the wall of a blood vessel, and one or more catheters or other treatment devices may be advanced therethrough into the vasculature.

Some endovascular procedures, especially those designed to treat the heart or large blood vessels such as the aorta, may require large-French vascular access. For example, treatment devices used in endovascular aneurysm repair procedures (treating abdominal or thoracic aortic aneurysms by delivery of a stent graft or other graft thereto) and endovascular aortic valve replacement generally range in size from about 12 Fr (about 4 mm) to about 30 Fr (about 10 mm). Accordingly, any vascular access point (e.g., the arteriotomy or other vessel opening) must be large enough to accommodate these large-French treatment devices, and thus vascular access is usually obtained through the common femoral artery or one of the iliac arteries (e.g., the common iliac artery, the external iliac artery, or the internal iliac artery).

The relatively large size of the treatment devices and associated vascular access may carry an increased risk of vessel perforations or bleeding events (e.g., resulting from incomplete or failed closure of the large-French openings). These bleeding events may be very time sensitive, with uncontrolled bleeding potentially leading to serious complications, such as hypovolemic shock, renal failure, consumption coagulopathy, limb loss, brain damage, or even death. Accordingly, it may be desirable to provide improved systems and methods for controlling bleeding that may result from an endovascular procedure.

### BRIEF SUMMARY

Described here are systems and methods for controlling bleeding. In some variations, a blood control system may comprise a blood control catheter, wherein the blood control catheter comprises an expandable member on a distal end of the blood control catheter. The systems may additionally comprise an intra-vessel support delivery catheter sized and configured to be slidably received in the blood control catheter, and an intra-vessel support connected to the intra-vessel support delivery catheter by a retrieval mechanism. The intra-vessel support may be moveable between an unexpanded configuration in which the intra-vessel support is positioned inside of the intra-vessel support delivery catheter and an expanded configuration in which the intra-vessel support is delivered from the intra-vessel support delivery catheter. The retrieval mechanism may be configured to retract the intra-vessel support from the expanded configuration to the unexpanded configuration. In some variations the intra-vessel support may be a stent graft, stent, cylindrical or other flow-through balloon. In some variations, the retrieval mechanism may be a tether. In other variations, the retrieval mechanism may be a catheter or sheath.

In some variations, the blood control system may further comprise a guidewire. The guidewire may be configured rapid-exchange or over-the-wire use. In some variations, the guidewire may comprise a core wire and a flexible sheet attached to the core wire. In some of these variations, the guidewire may further comprise an outer shaft, wherein the outer shaft is moveable between an advanced position to cover the flexible sheet and a retracted position to at least partially expose the flexible sheet. The flexible sheet may be configured such that is has a first width when covered by the outer shaft and a second width when exposed from the outer shaft, wherein the second width is larger than the first width. In some variations, the core wire may comprise a distal portion ending distally from a distal end of the flexible sheet. In some of these variations, the distal portion of the core wire may be configured to be radiopaque.

Also described here are methods of performing an endovascular procedure. In some variations, the method may comprise forming a first access site in a first blood vessel and advancing a first access sheath into the first blood vessel through the first access site. In some variations, the first access site may be formed in a common femoral artery, a common iliac artery, an external iliac artery or an internal iliac artery. In other variations, the first access site may be formed in a radial artery, a brachial artery, a subclavian artery, a carotid artery, or the like. In some variations, the first access sheath may be at least 12 French in diameter. The method may further comprise forming a second access site in a second blood vessel, wherein the second blood vessel is contralateral to the first blood vessel and advancing a second access sheath into the second blood vessel through the second access site. In some variations, the second first access site may be formed in a common femoral artery, a common iliac artery, an external iliac artery or an internal iliac artery. In other variations, the second access site may be formed in a radial artery, a brachial artery, a subclavian artery, a carotid artery, or the like. In some variations, the first access sheath may have a larger diameter than the second access sheath. The method further may comprise advancing a blood control catheter through the second access sheath to position the blood control catheter contralaterally to the first access site, and performing an endovascular procedure through the first access sheath while the distal end of the blood control catheter is positioned contralaterally to the first access site.

In some variations, the blood control catheter may comprise an expandable member. In some of these variations, the method may comprise advancing the blood control catheter to position the expandable member ipsilaterally and upstream of the first access site, and expanding the expandable member to occlude blood flow past the expandable member. In some of these variations, the method may further comprise withdrawing the first access sheath through the first access site, and closing the first access site. In some of these variations, the method may further comprise moving the expandable member to an unexpanded configuration and checking for bleeding.

Also described here are methods of controlling bleeding at a vascular opening in a first blood vessel. In some variations, the method may comprise introducing a blood control catheter into an access site in a second blood vessel, the blood control catheter comprising an expandable member. The first access site may be formed in a common femoral artery, a common iliac artery, an external iliac artery or an internal iliac artery. The method may comprise advancing the blood control catheter to position the expandable member upstream of the vascular opening, and expanding the expandable member to occlude blood flow past the expandable member. In some variations, the method may comprise advancing an intra-vessel support delivery catheter from a lumen of the blood control catheter, and delivering an intra-vessel support into the first blood vessel to cover the vascular opening, wherein a retrieval mechanism connects the intra-vessel support to the intra-vessel support delivery catheter.

In some variations, the method may further comprise returning the expandable member to an unexpanded configuration following delivery of the intra-vessel support. In some of these variations, the method may further comprise retrieving the intra-vessel support into the intra-vessel support delivery catheter. In some variations, the intra-vessel may comprise a stent graft. In some variations, the method may further comprise advancing a covered-stent delivery device through a second access site downstream of the vascular opening. In some of these variations, the method may further comprise retrieving the intra-vessel support into the intra-vessel support delivery catheter and delivering a covered-stent from the covered-stent delivery device to cover the vascular opening.

### BRIEF DESCRIPTION OF THE DRAWINGS

FIG. 1 shows an illustrative depiction of some of the major arteries of the abdomen and legs.

FIGS. 2A-2C depict an illustrative method of performing an endovascular procedure.

FIG. 3 depicts examples of possible bleeding sites that may occur during or after the endovascular procedure shown in FIGS. 2A-2C.

FIGS. 4A and 4B depict distal portions of illustrative variations of the blood control systems described here.

FIGS. 5A and 5B depict top views of a variation of a guidewire suitable for use in the systems and methods described here. FIGS. 5C and 5D depict cross-sectional front views of the guidewire of FIGS. 5A and 5B.

FIG. 6 depicts the blood control system of FIG. 4A positioned in the vasculature.

FIGS. 7A-7C depict an illustrative variation of a method of positioning the guidewire of FIGS. 5A-5D in the vasculature.

FIGS. 8A-8D depict an illustrative method of performing an endovascular procedure. FIGS. 8E and 8F depict an illustrative method of controlling bleeding at a bleeding site.

FIGS. 9A-9D depict an illustrative method of controlling bleeding at a vasculature perforation.

### DETAILED DESCRIPTION

Described here are systems and methods for controlling bleeding. The systems and methods may be used to control bleeding that occurs during or resulting from an endovascular procedure. In other instances, the systems and methods may be used to control bleeding in trauma patients in instances where a vessel perforation or other bleed site is suspected or detected, but which cannot be immediately addressed. The systems and methods described here may be used to quickly stop bleeding, and in some instances may stop bleeding without blocking blood flow through the vasculature for an extended period of time.

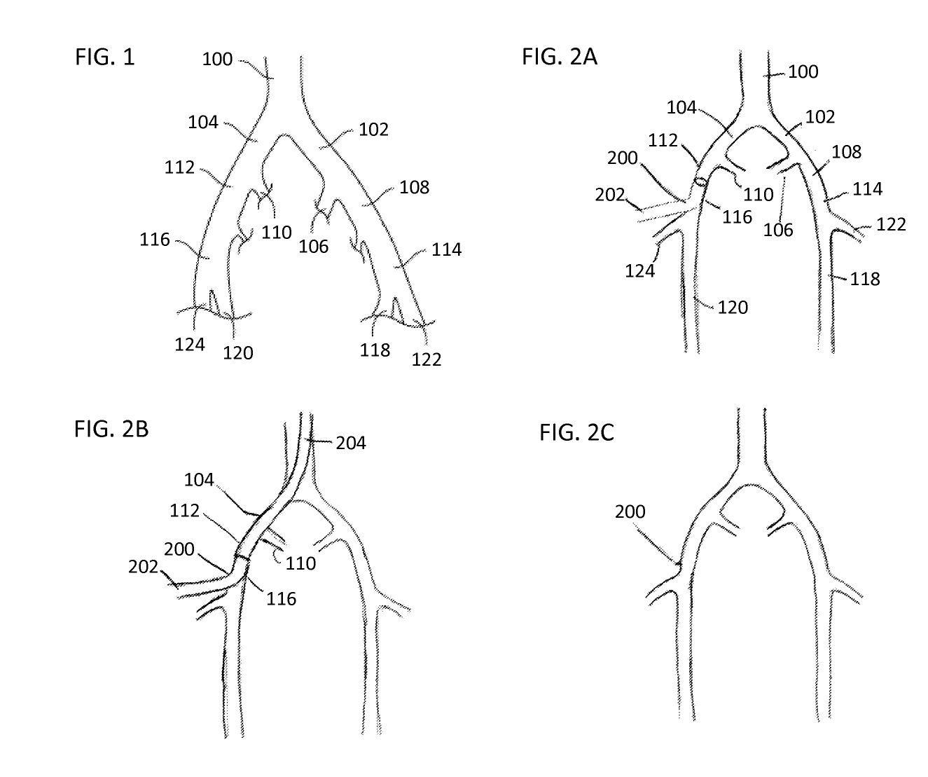

To help in understanding the systems and methods described here, FIG. 1 shows an illustrative depiction of some of the major arteries of the abdomen and legs. As shown there, the abdominal aorta (100) bifurcates around the level of the fourth lumbar vertebrae (not shown) into the left (102) and right (104) common iliac arteries. The left common iliac artery (102) later bifurcates into the left internal iliac artery (106) and the left external iliac artery (108). Similarly the right common iliac artery bifurcates into the right internal iliac artery (110) and the right external iliac artery (112). At or near the right and left inguinal ligaments (not shown) in the pelvis, the left (108) and right (112) external iliac arteries continues into the left (114) and right (116) common femoral arteries, respectively. Each of the common femoral arteries bifurcates into the deep femoral artery (labeled as (118) for the left and (120) for the right) and the superficial femoral artery (labeled as (122) for the left and (124) for the right). When these blood vessels are depicted in other figures described here, the blood vessels will be labeled as they are in FIG. 1.

As mentioned above, the systems and methods described here may be used to control bleeding during a percutaneous endovascular procedure. Some of these endovascular procedures, especially those designed to treat the heart or large blood vessels such as the aorta, may require large-French vascular access. For example, treatment devices used in endovascular aneurysm repair procedures (treating abdominal or thoracic aortic aneurysms by delivery of a stent graft or other graft thereto) and endovascular aortic valve replacement generally range in size from about 12 Fr (about 4 mm) to about 30 Fr (about 10 mm). Accordingly, any vascular access point (e.g., an arteriotomy or other vessel opening) must be large enough to accommodate these large-French treatment devices, and thus vascular access is usually obtained through the common femoral artery or one of the iliac arteries (e.g., the common iliac artery, the external iliac artery, or the internal iliac artery).

FIGS. 2A-2C depicts an illustrative example of a method of performing an endovascular procedure. As shown there, the method may include creating an endovascular procedure (“EVP”) access site (200) in a blood vessel, and advancing an access sheath (202) into the blood vessel through the access site (200). The access site (200) may be an arteriotomy or other opening formed in the vessel. While shown in FIG. 2A as being formed in a common femoral artery (e.g., the right common femoral artery (116) as shown in FIG. 2A or the left common femoral artery (114), the access site (200) may alternatively be formed in a common iliac artery (e.g., the right (104) or left (102) common iliac arteries), an external iliac artery (e.g., the right (110) or left (108) external iliac arteries), or an internal iliac artery (e.g., the right (110) or left (106) internal iliac arteries). While the method depicted in FIGS. 2A-2C is shown as being performed through blood vessels of the right side of the body, the endovascular procedure may alternatively be performed through the blood vessels of the left side of the body. In other instances, one or more endovascular procedures may be performed through an access site in a radial artery, a brachial artery, a subclavian artery, a carotid artery, or the like.

With the access sheath (202) in place through the access site (200), one or more EVP treatment devices may be introduced into the vasculature through the access sheath (202) to perform an endovascular procedure. For example, FIG. 2B shows a first EVP treatment device (204) extending from the access sheath (202) and advanced into the aorta (100). To reach the aorta (100), the first EVP treatment device (204) may be advanced through the blood vessels between the access site (200) and the aorta (100) (e.g., the right common iliac artery (104), the right external iliac artery (112) and the right common femoral artery (116), in instances where the access site (200) is formed in the right common femoral artery (116)). In some instances, the EVP treatment device (204) may be advanced from the aorta (100) into the heart (not shown) and/or the vasculature of the upper body (e.g., a radial, a carotid, subclavian, or brachial artery), depending on the target treatment locations of the endovascular procedure.

During the endovascular procedure, any number of EVP treatment devices may be introduced into and/or removed from the vasculature through the access sheath (202) as may be necessary to perform the endovascular procedure. The endovascular procedure may be any suitable endovascular procedure, such as, for example, an aneurysm repair procedure, a percutaneous heart valve replacement or repair procedure (e.g., a mitral valve procedure, an aortic valve procedure, or the like), a closure procedure to close or occlude one or more structures (e.g., the patent foramen ovale, the left atrial appendage, an apical access point, etc.), a structural heart procedure to treat congestive heart failure, or the like, and the EVP treatment devices may be any suitable device configured to assist in the percutaneous procedure (e.g., a stent or stent-graft delivery device, a balloon valvuloplasty device, or the like).

Once the endovascular procedure has been completed, the EVP treatment devices and the access sheath (202) may be removed from the access site (200), and the access site (200) may be closed, such as illustrated in FIG. 2C. Generally, the access site (200) may be closed using one or more sutures, clips, or other similar elements. In some instances, a pressure dressing and/or sandbag may be applied to the patient to help maintain hemostasis.

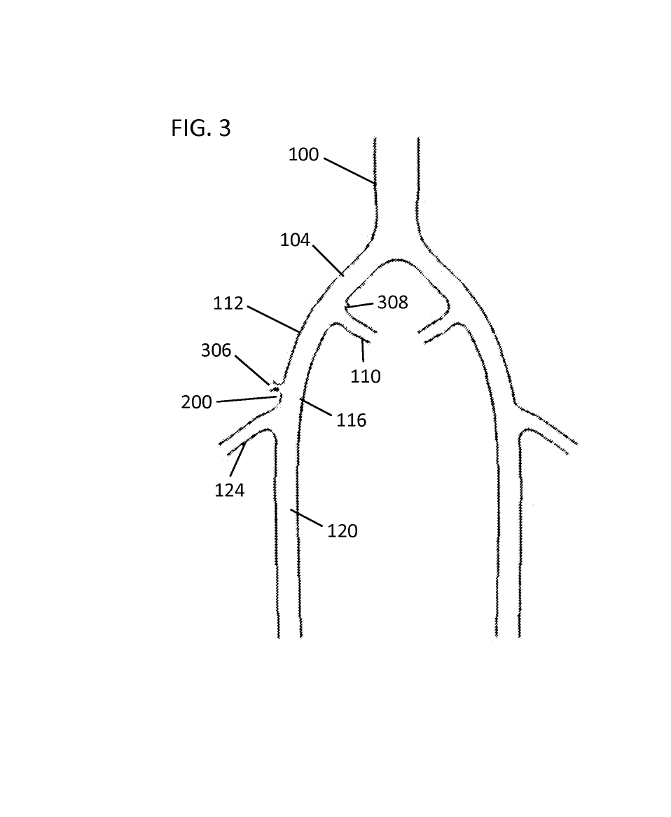

In some instances, one or more injuries may occur during or after the endovascular procedure, which may result in bleeding. FIG. 3 shows two possible injuries which may occur during or after the endovascular procedure depicted in FIGS. 2A-2C. In some instances, the closed access site (200) may subsequently re-open, creating a bleed site (306). Such a bleed site (306) may occur when the suture, clip, or other element used to close the access site (200) tears through the vessel wall and no longer holds the access site (200) closed.

In other instances, advancement/tracking of the access sheath (202) or one of the EVP treatment devices (e.g., the first EVP treatment device (204)) in the vasculature may damage a blood vessel, thereby creating a vessel tear (308). These vascular tears can be quite common, as the size of the procedural devices may be large relative to the size of the vasculature and the procedural devices typically have a level of stiffness that may result in transmission of pushing forces from the procedural device to the vessel wall. While shown in FIG. 3 as being formed in the common iliac artery (104), a vessel tear (308) may form in any suitable vessel (e.g., an external iliac artery, an internal iliac artery, a common femoral artery). In other instances, a vessel tear may occur in a radial artery, a brachial artery, a subclavian artery, a carotid artery, or the like.

Either a vessel tear (308) or a bleed site (306) formed in a closed access site (200) may result in hematoma or blood loss, which, if not controlled quickly, may lead to serious complications, such as hypovolemic shock, renal failure, consumption coagulopathy, limb loss, brain damage, or even death. Accordingly, it may be desirable to stop this blood loss quickly. Accordingly, the systems and methods described here may be configured to control blood loss in the instance of vessel tear (308) or access site bleed site (306).

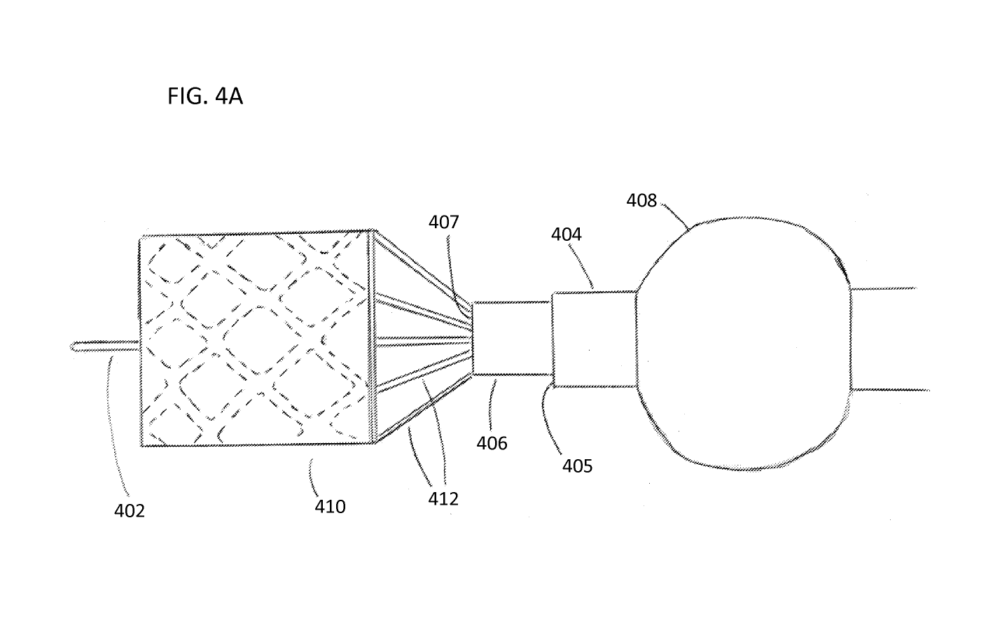

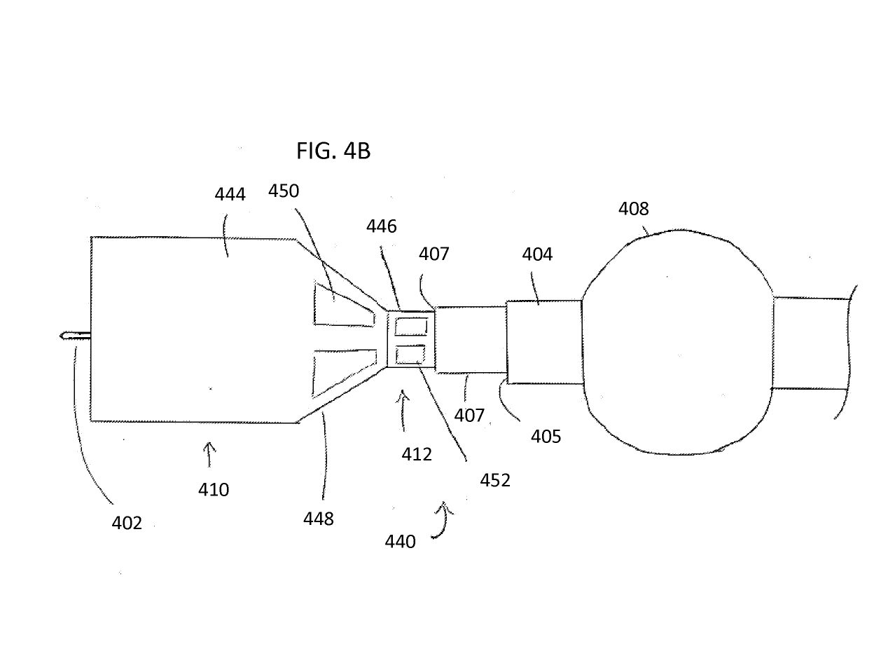

FIG. 4A shows a variation of a blood control system (400) as described here which may be used to control bleeding during or after an endovascular procedure (although it should also be appreciated that the blood control system (400) may be used to control bleeding in any suitable instance, which need not be in the context of an endovascular procedure. As shown in FIG. 4A, the blood control system (400) may comprise a guidewire (402), a blood control catheter (404), and an intra-vessel support (“IVS”) delivery catheter (406). As shown there, the IVS delivery catheter (406) may include a lumen (407) extending at least partially therethrough, such that the guidewire (402) may be slidably received in the lumen (407) to allow the WS delivery catheter (406) to be advanced along the guidewire (402). Similarly, the blood control catheter (404) may comprise a lumen (405) extending at least partially therethrough, such that at least a portion of the guidewire (402) may be slidably received in the lumen (405) to allow the blood control catheter (404) to be advanced along the guidewire (402). Additionally, the lumen (405) of the blood control catheter (404) may be sized such that at least a portion of the IVS delivery catheter (406) may be positioned in the lumen (405). This may allow a distal portion of the IVS delivery catheter (406) to be advanced out of a distal portion of the blood control catheter (404), such as shown in FIG. 4A.

Generally, the blood control catheter (404) may comprise an expandable member (408) which may be selectively moved between an expanded and an unexpanded configuration. When the expandable member (408) is expanded in a blood vessel, the expandable member (408) may substantially occlude the vessel, which may prevent blood flow past the expandable member (408). The expandable member (408) may be any suitable expandable structure. For example, in the variation of blood control catheter (404) shown in FIG. 4A, the expandable member (408) may comprise a balloon. The balloon may be selectively inflated or deflated (e.g., via an inflation line) to expand and contract, respectively, the balloon.

As mentioned above, the IVS delivery catheter (406) may be sized for advancement through the blood control catheter (404). The IVS delivery catheter (406) may be configured to temporarily deliver an intra-vessel support (410) into the vasculature. The intra-vessel support (410) may be configured to temporarily control bleeding out of a bleed site, as will be described in more detail below. The IVS delivery catheter (406) may be configured such that the intra-vessel support (410) may be delivered from the IVS delivery catheter (406) into the vasculature, and may be retrieved from the vasculature to return the intra-vessel support (410) to the IVS delivery catheter (406). For example, in the variation shown in FIG. 4A, the IVS delivery catheter (406) may comprise a retrieval mechanism (412) configured to connect the intra-vessel support (410) to the IVS delivery catheter (406).

The retrieval mechanism (412) may be any mechanism suitable to withdraw the intra-vessel support (410) into the lumen (407) of the IVS delivery catheter (406), such as a tether. In some variations, the retrieval mechanism (412) may be configured to be severable such that the connection between the IVS delivery catheter (406) and the intra-vessel support (410) may be severed. For example, the tether may be configured to be released via application of an electrical current to the tether, or via one or more release mechanisms such as a latch, pull-string or filament with may be withdrawn relative to the retrieval mechanism (412) to sever the connection between the retrieval mechanism (412) and the intra-vessel support (410).

Generally, the intra-vessel support (410) may comprise an expandable tubular member which may be moveable between an unexpanded and an expanded configuration. The intra-vessel support (410) may be configured to fit inside the WS delivery catheter (406) when in the unexpanded configuration. When delivered from the IVS delivery catheter (406) (such as shown in FIG. 4A), the intra-vessel support (410) may expand to the expanded configuration within a blood vessel. As the intra-vessel support expands, the intra-vessel support (410) may press against an interior of the blood vessel. In some instances, the wall of the intra-vessel support may cover a vessel perforation or bleed site (as will be described in more detail below), and may thereby prevent blood from exiting the blood vessel via the bleed site. Additionally, blood may continue to flow through the tubular intra-vessel support (410).

The intra-vessel support (410) may self-expand from its unexpanded configuration to its unexpanded configuration, or may be expandable using another device (such as a balloon which may be positioned within the intra-vessel support (410)). When delivered from the IVS delivery catheter (406), the retrieval mechanism (412) may maintain a connection between the IVS delivery catheter (406) and the intra-vessel support (410). The intra-vessel support (410) may be returned to an unexpanded configuration by withdrawing the retrieval mechanism (412) into the IVS delivery catheter (406), which in turn may pull the intra-vessel support (410) into the IVS delivery catheter (406). The intra-vessel support (410) may be any suitable structure, such as, for example, a covered stent/stent graft (such as shown in FIG. 4A) or a tubular inflatable balloon. In variations where the intra-vessel support (410) comprises a stent graft, the stent may comprise a self-expanding stent (e.g., a NiTi stent, metallic braid or the like) with a non-permeable covering. The non-permeable covering may be integrally formed with the self-expanding stent, or may be applied and affixed to the inner and/or outer surfaces of the self-expanding stent.

FIG. 4B shows another variation of a blood control system (440) as described here. As shown there, the blood control system (440) may comprise a guidewire (402), a blood control catheter (404), and an IVS delivery catheter (406) such as described in more detail above. The IVS delivery catheter (406) may be configured to deliver an intra-vessel support (410) which may be connected to the delivery catheter (406) via a retrieval mechanism (412). In the variation shown in FIG. 4B, the intra-vessel support (410) may comprise a stent graft (444), and the retrieval mechanism (412) may comprise a retrieval catheter (446). The retrieval catheter (446) may be slidably received in a lumen of the IVS delivery catheter (406) to allow the retrieval catheter (446) to be advanced and retracted relative to the IVS delivery catheter (406), and may comprise a lumen extending therethrough such that the retrieval catheter (446) may be advanced along the guidewire (402). A distal portion of the retrieval catheter (446) may be releasably or permanently connected to a proximal portion of the stent graft (444), such that advancement of the retrieval catheter (446) may push the stent graft (444) out of the IVS delivery catheter (406) and withdrawal of the retrieval catheter (446) may pull the stent graft (444) back into the WS delivery catheter (406). In some variations, a pull wire or the like may releasably connect the stent graft (444) and the retrieval catheter (446), such that the stent graft (444) may be released from the retrieval catheter (446). In other variations, the stent graft (444) may be permanently connected to the retrieval catheter (446).

When the stent graft (444) is positioned outside of the IVS delivery catheter (406), the diameter of the stent graft may expand to an expanded configuration, such as described above. When in the expanded configuration, the stent graft (444) may comprise a tapered segment (448) which may have a diameter that transitions between the expanded diameter of the stent graft (444) and the diameter of the retrieval catheter (446). This tapered diameter may aid in retrieval of the stent graft (444) back into the IVS delivery catheter (406). Additionally, as shown in FIG. 4B, the stent graft (444) and/or the retrieval catheter (446) may comprise one or more apertures extending therethrough, which may allow blood to pass through the lumen of the stent graft (444) when the stent graft (444) is expanded in the vessel. For example, the stent graft (444) may comprise one or more apertures (450) extending through a wall of the stent graft (444). In some of these variations, the apertures (452) may extend through the tapered segment (448). Additionally or alternatively, the retrieval catheter (446) may comprise one or more apertures (452) extending through a wall of the retrieval catheter (446). While shown in FIG. 4B as having both one or more apertures (450) extending through the stent graft (444) and one or more apertures (452) extending through the retrieval catheter (446), in other variations only one of the stent graft (444) or retrieval catheter (446) comprises apertures extending therethrough. In still other variations, neither the stent graft (444) nor the retrieval catheter (446) may comprise an aperture extending therethrough.

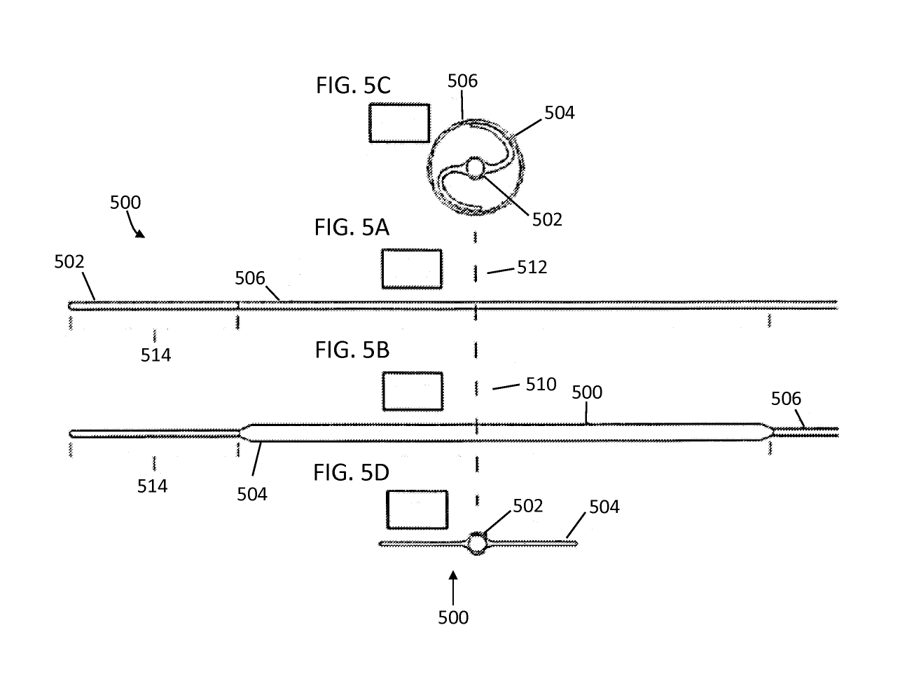

The guidewire (402) may be any guidewire suitable for introduction into and advancement through the vasculature. For example, FIGS. 5A-5D illustrate one variation of a guidewire (500) suitable for use with the blood control systems described here (although it should be appreciated that the guidewire (500) may be used in any suitable procedure with any suitable devices). FIGS. 5A and 5B show top views of the guidewire (500). As shown there, the guidewire (500) may comprise a core wire (502), a flexible sheet (504) attached to the core wire (502), and an outer shaft (506). The outer shaft (506) may be a tubular body having a lumen extending at least partially therethrough, and the core wire (502) may be slidably positioned at least partially in the lumen of the outer shaft (506).

The outer shaft (506) may be movable between an advanced, position (as shown in FIG. 5A) and a retracted position (as shown in FIG. 5B) to change the shape of the flexible sheet (504) from a first tracking configuration to a second delivery configuration, respectively. When the outer shaft (506) is moved to the advanced position shown in FIG. 5A, the outer shaft (506) may cover the flexible sheet (504) and place the flexible sheet (504) in the first configuration. When in the first configuration, the flexible sheet (504) may at least partially fold or wrap around the core wire (502), such as shown in a cross-sectional front view in FIG. 5C (taken through the line (512) shown in FIG. 5A). Because the flexible sheet (504) is held within the outer shaft (506), the maximum width that may be achieved by the flexible sheet (504) is that of the diameter of the outer shaft (506), and the guidewire (500) may have a substantially circular outer diameter, similar to that of conventional guidewires. This may allow the guidewire (500) to be advanced and tracked like a conventional guidewire. In some variations, the outer shaft (506) may have a diameter of out about 1 mm (or between about 0.5 mm and about 1.5 mm), while the unfolded flexible sheet (504) may have a width between about 3 mm and about 8 mm (preferably between about 4 mm and about 5 mm). In these variations, movement of the outer shaft (506) may move the width of the flexible sheet between about 1 mm (when constrained by the outer shaft (506)) to between about 3 mm and about 8 mm (when exposed from the outer shaft (506))

Conversely, when the outer shaft (506) is retracted to the position shown in FIG. 5B, at least a portion of the flexible sheet (504) may be exposed beyond a distal end of the outer shaft (506). When exposed from the outer shaft (506), the flexible sheet (504) may take on a second configuration having a substantially flattened shape, such as shown in FIG. 5B and in a cross-sectional front view in FIG. 5D (taken through line (510) in FIG. 5B). As shown in FIGS. 5B and 5D, the flattened flexible sheet (504) may have a width greater than the outer diameter of the outer shaft (506). The outer shaft (506) may be selectively advanced and retracted relative to the core wire (502) to move the flexible sheet (504) between the first and second configurations, respectively.

In use, the outer shaft (506) may be placed in the advanced position to place the guidewire (500) in a tracking configuration, and the guidewire (500) may be tracked through the vasculature. When the guidewire (500) has been positioned at a target location, the outer shaft (506) may be withdrawn to expose at least a portion of the flexible sheet (504), which may cause the flexible sheet (504) to move to the second configuration. When the flexible sheet (504) is flattened, the increased width of the flexible sheet (504) relative to the diameter of the outer shaft (506) may provide a larger surface area that may contact a vessel wall, which may in turn reduce the likelihood that the guidewire (500) may damage vessel walls during an endovascular procedure. For example, when a conventional guidewire is positioned between an interventional device (such as an access sheath and/or an EVP treatment device) and a vessel wall, pressure applied to the guidewire by the interventional device may cause the guidewire to perforate the vessel, thereby creating a bleeding complication. With the guidewire (500), however, the increase surface area provided by the flattened flexible sheet (504) may distribute pressure from an interventional device across a larger portion of the blood vessel, which in turn may reduce the likelihood of perforation. Additionally, the pressure applied to the flattened flexible sheet (504) may help to fixate the guidewire (500) relative to the blood vessel.

The core wire (502) and flexible sheet (504) may be made from any suitable material or combinations of materials (e.g., stainless steel, nitinol, cobalt chrome, or the like). The flexible sheet (504) may be formed integrally with the core wire (502), or may be formed separately from the core wire (502) and attached thereto. The flexible sheet (504) may be positioned along any suitable length of the core wire (502) (e.g., greater than about 10 cm, greater than about 30 cm, or the like). In some variations, the core wire (502) may comprise a distal segment (514) extending distally of the flexible sheet (504), but need not. In these variations, the distal segment (514) may maintain its shape during advancement and retraction of the outer sheath (506). In some of these variations, the distal segment (514) may be configured to be radiopaque, such that the distal segment (514) may be viewed via indirect visualization (e.g., via fluoroscopy). For example, the distal segment (514) of the core wire (502) may be formed from one or more radiopaque materials, may include a radiopaque wire attached to the core wire (502) (e.g., a helically coiled radiopaque wire), and/or may include a radiopaque coating (e.g., a radiopaque polymer coating or the like). Having a radiopaque distal segment (514) may help guide advancement of the guidewire (500), and may further allow a user to determine where the flexible sheet (504) has been positioned. In variations where the guidewire (500) is used with the blood control systems described here, it should be appreciated that in some instances, the guidewire (500) need not comprise an outer sheath (506), and one or more portions of the blood control system may be configured to temporarily constrain the flexible sheet (504). For example, in some variations where a blood control system comprises a retrieval mechanism that includes a catheter, the catheter may be configured to have an inner diameter that may constrain the flexible sheet (504). In these variations, the guidewire (500) may be advanced relative to the retrieval mechanism to expose a portion of the flexible sheet (504).

As mentioned above, the systems described here may be used to control bleeding during or after an endovascular procedure. The systems described here may be used to temporarily control bleeding at a bleed site that forms at a previously closed access site (such as bleed site (306) shown in FIG. 3 above) or at the location of a vessel tear (e.g., a vessel tear formed during the endovascular procedure, such as the vessel tear (308) shown in FIG. 3 above), as the need may arise. Methods of temporarily controlling bleeding at each of these possible bleed locations will be described below.

To use the blood control systems described here to control bleeding during or after an endovascular procedure, the blood control system may first be positioned in the vasculature. In some variations, the blood control system may be positioned in the vasculature prior to beginning the endovascular procedure, such that the blood control system is positioned in the vasculature during the endovascular procedure. In other variations, the blood control system may be positioned in the vasculature during the endovascular procedure. In yet other variations, the blood control system may be positioned in the vasculature after the endovascular procedure has been completed.

When an endovascular procedure is performed through an access site in a blood vessel on a first of a patient, the blood control system may be introduced through a contralateral blood vessel. Generally, when the terms “contralateral” and “ipsilateral” are used here, they are used to discuss blood vessels in relation to an EVP access site (i.e., the ipsilateral vessels are those one the same side of the body as the EVP access site, and the contralateral vessels are those on the opposite side of the body as the EVP access site). It should also be appreciated that in some instances the blood control system may be introduced from an access point in the upper body (e.g., a brachial artery) and may be introduced through an access point in a radial artery, a brachial artery, a subclavian artery, a carotid artery, or the like

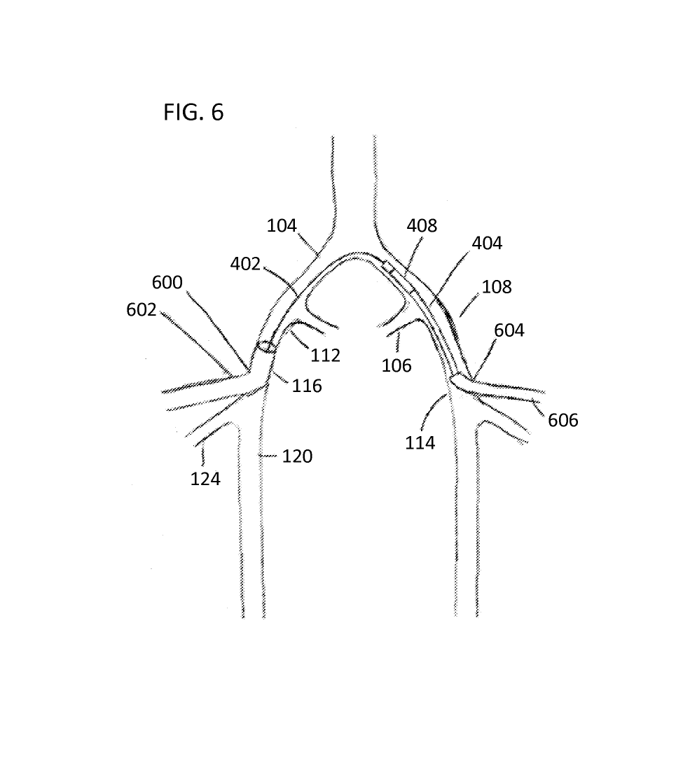

For example, FIG. 6 shows the blood control system (400) (described above with respect to FIG. 4A) positioned relative to an EVP access site (600). While shown in FIG. 6 as being formed in the right common femoral artery (116), it should be appreciated that the EVP access site (600) may be formed in any suitable blood vessel, such as described above with respect to FIGS. 2A-2C. An EVP access sheath (602) may be introduced into the vasculature via the EVP access site (600), which may provide an access route for the introduction of one or more EVP treatment devices to perform an endovascular procedure, such as discussed above.

As shown in FIG. 6, the blood control system (400) may be introduced through a blood control (BC) access site (604) in a contralateral blood vessel. The BC access site (604) is shown in FIG. 6 as being formed in the left common femoral artery (114), but it should be appreciated that the BC access site (604) may be formed in any suitable contralateral vessel (e.g., a contralateral common iliac artery, external iliac artery, internal iliac artery, or common femoral artery). In some variations, a BC access sheath (606) may be positioned in the contralateral vasculature through the BC access site (604), through which one or more components of blood control system (400) may be introduced into the vasculature. The BC access sheath (606) may be any suitable size (e.g., between 6 French and 9 French), and may in some instances be smaller than the EVP access sheath (602).

For example, the guidewire (402) may be introduced through the BC access site (604) such that it traverses the connection of the common iliac arteries between the contralateral and ipsilateral blood vessels. A distal portion of the guidewire (402) may be positioned inside the EVP access sheath (602), or may be positioned between the EVP access sheath (602) and a vessel wall. The guidewire (402) may create a track between the BC access site (604) and the EVP access sheath (602), which may allow other components of the blood control system (400) to be guided from the BC access site (604) into the vasculature. Specifically, the blood control catheter (404) may be advanced into the vasculature over the guidewire (402). The blood control catheter (404) may be positioned entirely in the contralateral vasculature (as shown in FIG. 6), or may be advanced into the ipsilateral vasculature, as will discussed in more detail below.

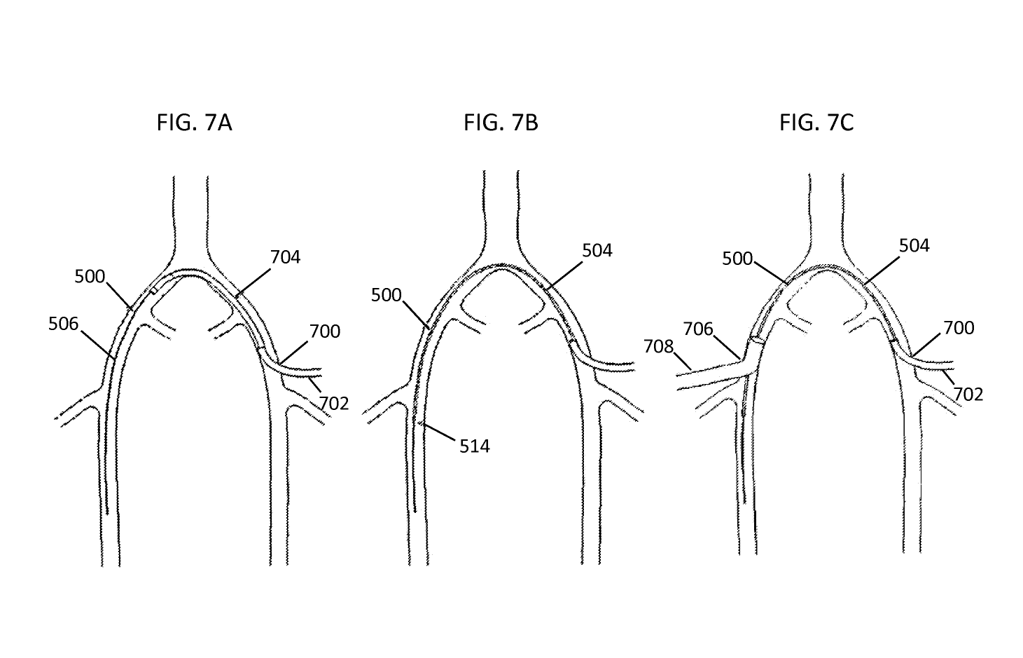

When a guidewire (402) is advanced through a BC access site, across the common iliac arteries, and into or next to an access sheath (602) (such as shown in FIG. 6), the guidewire (402) may be introduced into the body before or after formation of the EVP access site (600) and/or insertion of the EVP access sheath (602) into the vasculature. For example, FIGS. 7A-7C depict one method of positioning a guidewire relative to an EVP access site and EVP access sheath. While these figures depict a method of delivering the variation of the guidewire (500) discussed above with respect to FIGS. 5A-5D, it should be appreciated that these methods may be used to position any suitable guidewire. As shown in FIG. 7A, a BC access site (700) may be formed in a contralateral blood vessel (the BC access site (700) may be formed in any suitable blood vessel, such as described in more detail above), and an BC access sheath (702) and guidewire (500) may be introduced into the contralateral vasculature through the BC access site (700). In some variations, the guidewire (500) may be tracked with the aid of a support sheath (704), but need not be. In some variations, the EVP access site and/or the BC access sites may be formed in a radial artery, a brachial artery, a subclavian artery, a carotid artery, or the like.

As shown in FIG. 7A, the guidewire (500) may be advanced from the BC access site (700) through the contralateral vasculature, across the common iliac arteries, and into the ipsilateral vasculature. In some variations, the guidewire (500) may be advanced in a tracking configuration (i.e., with the outer sheath (506) in an advanced position to constrain the width of the guidewire (500)). Generally, the guidewire (500) may be advanced such that the distal end of the guidewire (500) is positioned distally (e.g., downstream) of the anticipated EVP access site. In some variations where the core wire (502) includes a distal segment (514) distal to the flexible sheet (504), the guidewire (500) may be advanced to position the entirety of the distal segment (514) distally of the anticipated EVP access site.

With the guidewire (500) positioned as discussed above, the outer sheath (506) may be retracted to expose the flexible sheet (504), as shown in FIG. 7B. In variations where a support sheath (706) is used to help guide the guidewire (500), the support sheath (704) may be also removed from the vasculature. As the flexible sheet (504) is exposed, the flexible sheet (504) may change to a delivery configuration, such as discussed above. In some variations, the flexible sheet (504) may be formed with such a length such that the exposed flexible sheet (504) may extend from a point distal to the anticipated EVP access site to the BC access sheath (702). With the guidewire (500) in place, an EVP access site (706) may be formed in an ipsilateral vessel (which may be any of the ipsilateral vessels described above), and an EVP access sheath (708) may be introduced into the ipsilateral vasculature through the EVP access site (706), such as shown in FIG. 7C. In some variations, the presence of the EVP access sheath (708) in the vasculature may push the guidewire (500) against a vessel wall. The pressure applied to the guidewire (500) may help to temporarily anchor the guidewire (500) in place relative to the EVP access sheath (708) (which may help facilitate advancement of other components of the blood control system over the guidewire (500)), but the extra surface area provided by the unfolded flexible sheet (504) may reduce the likelihood that guidewire (500) may perforate the vessel wall.

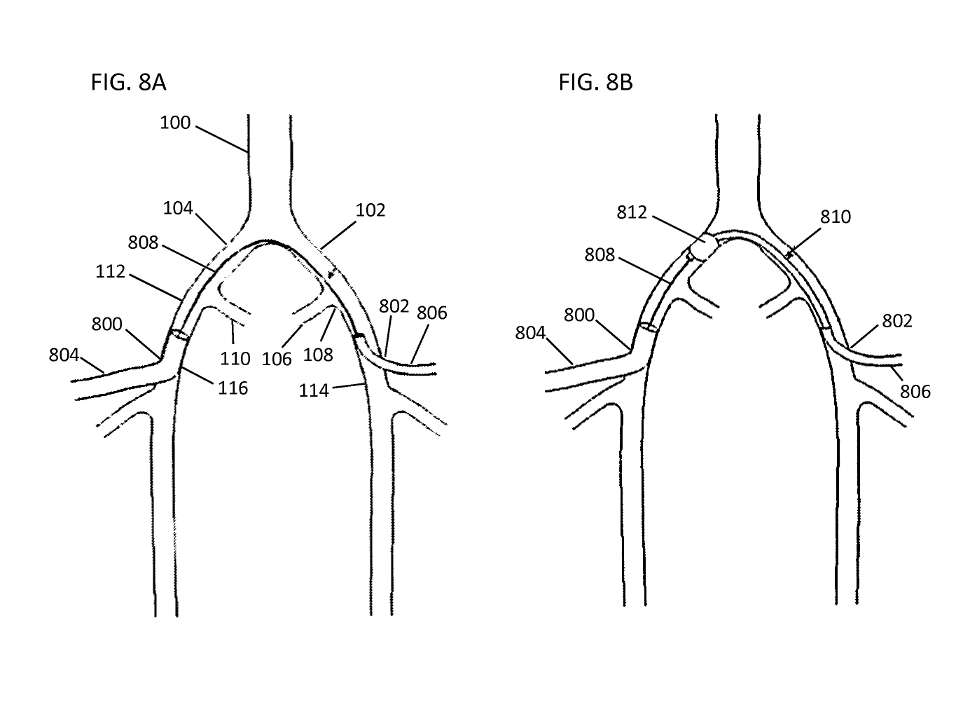

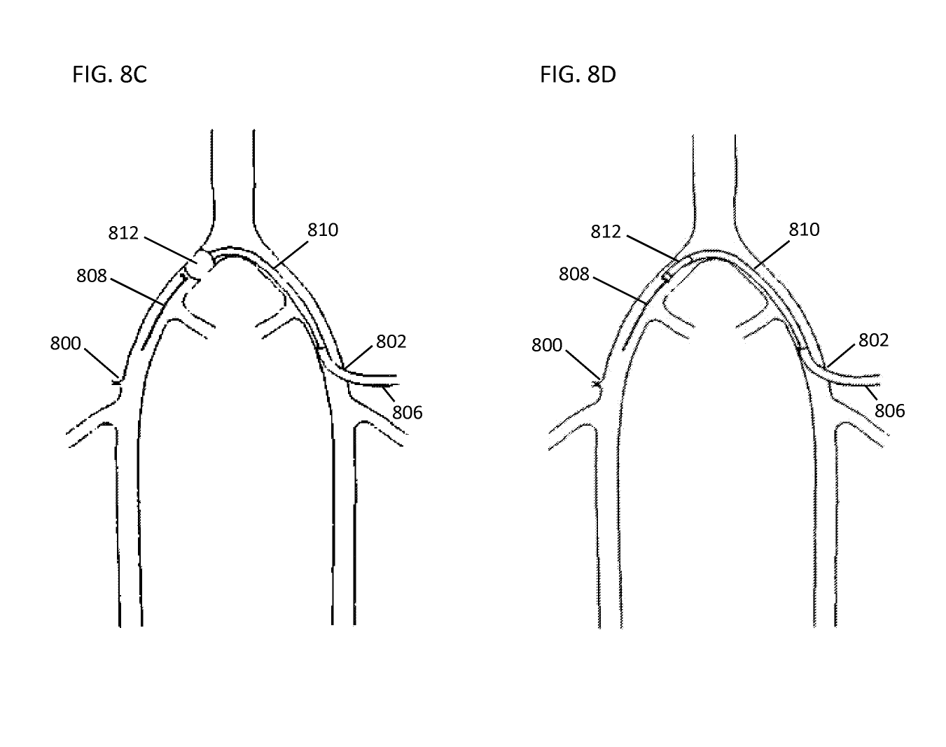

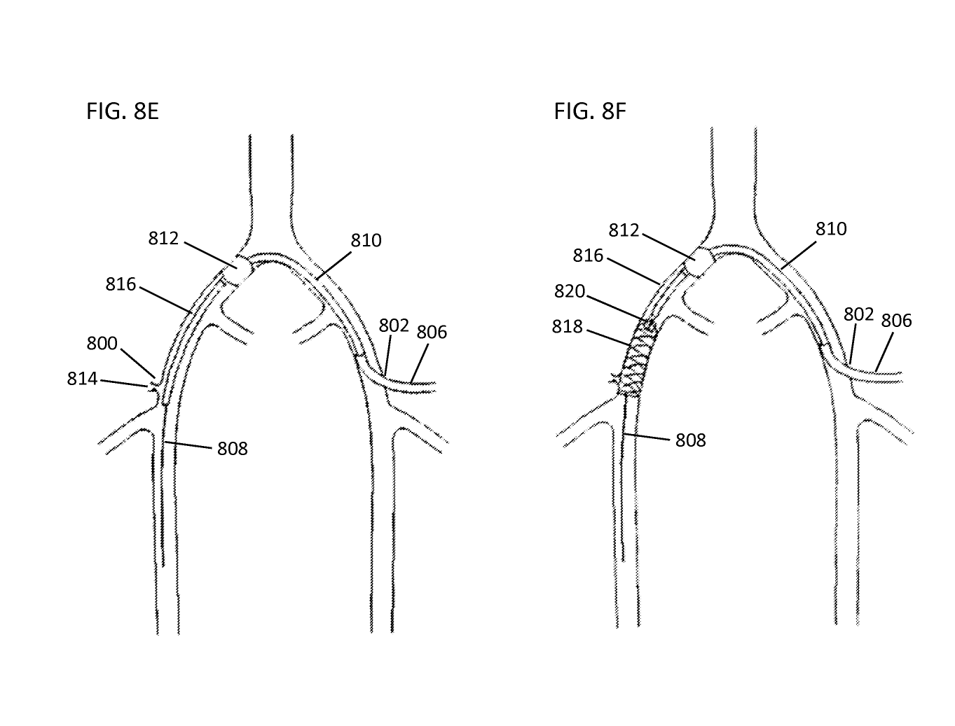

Once positioned, the blood control systems described here may be used to control bleeding in the occurrence of one or more bleeding events. For example, FIGS. 8A-8F depict a method of controlling bleeding in the instance of a bleed site formed at a previously closed access site using a blood control system. The blood control system may comprise a guidewire (808), a blood control catheter (810), and an IVS delivery catheter (816), such as those described above with respect to FIGS. 4A and 4B above. As shown in FIG. 8A, an EVP access site (800) and a BC access site (802) may be formed in an ipsilateral blood vessel and a contralateral blood vessel, respectively. These access sites may be formed in any combination of ipsilateral and contralateral blood vessels, such as discussed in more detail above. An EVP access sheath (804) may be advanced and positioned in the ipsilateral vasculature through the EVP access site (800), and a BC access sheath (806) may be advanced and positioned in the contralateral vasculature through the BC access site (802). Additionally, a guidewire (808) (which may be any suitable guidewire, such as those discussed above) may be advanced through the BC access site (802), across the common iliac arteries, and positioned such that distal portion of the guidewire (808) is positioned inside of the EVP access sheath (804) or between the EVP access sheath (804) and the vessel wall.

An endovascular procedure may then be performed using one or more EVP treatment devices (not shown) advanced through the EVP access site (800) and EVP access sheath (804), such as described above with respect to FIGS. 2A-2C. Following completion of the endovascular procedure, the EVP treatment devices may be removed from the vasculature via the EVP access sheath (804). In some variations, the blood control catheter (810) may be advanced over the guidewire (808) through the BC access site (806), and may be positioned such that distal end of the blood control catheter (810) is positioned in the contralateral vasculature during the endovascular procedure.

With the EVP treatment devices removed, the blood control catheter (810) may be advanced into the ipsilateral vasculature to position an expandable member (812) of the blood control catheter in an ipsilateral blood vessel upstream of the EVP access sheath, such as shown in FIG. 8B. In some variations, the expandable member (812) may be positioned in a common iliac artery. In other variations, the expandable member (812) may be positioned in an external iliac artery, an internal iliac artery, or a common femoral artery. The expandable member (812) may then be expanded to block blood flow past the expandable member (812), which may also block blood flow toward the EVP access sheath.

With blood flow blocked by the expandable member (812), the EVP access sheath (804) may be removed from the vasculature through the EVP access site (800), and the EVP access site (800) may be closed, as shown in FIG. 8C. The EVP access site (800) may be closed in any suitable manner, such as described in more detail above with respect to FIGS. 2A-2C. Once the EVP access site (800) is closed, the expandable member (812) of the blood control catheter may be unexpanded, such as shown in FIG. 8D, thereby allowing blood flow to resume past the expandable member (812). A practitioner may then check for bleeding sites. In some variations, this may comprise an external visual examination of the patient to check for bleeding. Additionally or alternatively, this may comprise introducing a fluoroscopic agent (e.g., a fluoroscopic die) into the ipsilateral vasculature (which may be introduced through a lumen of the blood control catheter (810)), and fluoroscopically visualizing the patient.

If no bleeding is detected, the blood control catheter (810), the guidewire (808), and the BC access sheath (806) may be removed from the vasculature through the BC access site (802), and the BC access site (802) may be closed using any suitable method. In some variations, the blood control catheter (810) may be left in place for a period of time (e.g., five minutes, fifteen minutes, thirty minutes, or the like) after the initial bleed check, and the patient may be periodically checked for bleeding events. If no bleeding events have occurred during that period, the blood control catheter (810) and other components may be removed and the BC access site (802) may be closed (e.g., using one or more sutures, clips, adhesives, or other closure techniques).

If a bleeding event is detected, the blood control system may be used temporarily control the bleeding. For example, if a bleeding check demonstrates that the a bleed site (814) has occurred at the previously-closed EVP access site (800), the expandable member (812) of the blood control catheter (810) may be re-expanded to again block blood flow past the expandable member (812), as shown in FIG. 8E. The IVS delivery catheter (816) may be advanced through the blood control catheter (810), as shown in FIG. 8E, and an intra-vessel support (818) (such as described in more detail above) may delivered from the IVS delivery catheter (816), such as shown in FIG. 8F. Upon delivery of the intra-vessel support (818), the intra-vessel support (818) may expand into contact with the vessel housing the bleed site (814), and may be positioned such that the intra-vessel support (818) covers the bleed site (814). A retrieval mechanism (820) may maintain a connection between the IVS delivery catheter (816) and the intra-vessel support (818) while the intra-vessel support (818) is positioned to cover the bleed site (814).

With the intra-vessel support (818) positioned as shown in FIG. 8F, the expandable member (812) of the blood control catheter (810) may be unexpanded to allow blood flow past the expandable member (812). As blood reaches the intra-vessel support (818) it may flow through a lumen of the intra-vessel support (818), thereby bypassing the bleed site (814). Accordingly, the intra-vessel support (818) may control bleeding from the bleed site (814) while still allowing blood flow through the ipsilateral vasculature. It should be appreciated that the retrieval mechanism (820) may be configured such that it does not prevent blood from passing by the retrieval mechanism (820). This may provide a physician additional time to reclose the EVP access site (800), which may be especially useful in instances where a physician may not attend to the bleeding site immediately.

When the EVP access site (800) is reclosed, the intra-vessel support (818) may be retrieved into the IVS delivery catheter (816) (e.g., by retracting the retrieval mechanism (820) relative to the IVS delivery catheter (816)). In some variations, this may comprise re-expanding the expandable member (812) of the blood control catheter (810), retracting the intra-vessel support (818) into the IVS delivery catheter (816), and contracting the expandable member (812). A user may again check for bleeding, and if no bleeding is found, the components of the blood control system may be removed from the vasculature via the BC access site (802). If bleeding is again found, the intra-vessel support (818) may be redelivered to control the bleed site, as discussed above. Additionally, in some variations, the intra-vessel support (818) may be disconnected from the IVS delivery catheter (816) to permanently deploy the intra-vessel support (818) in the vasculature, such as described in more detail above.

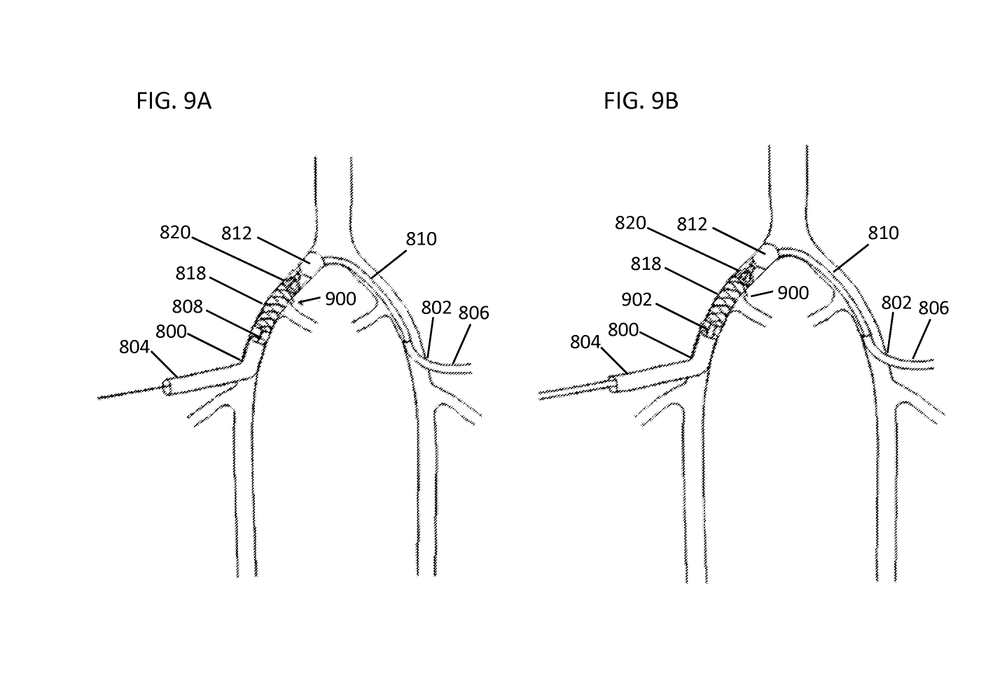

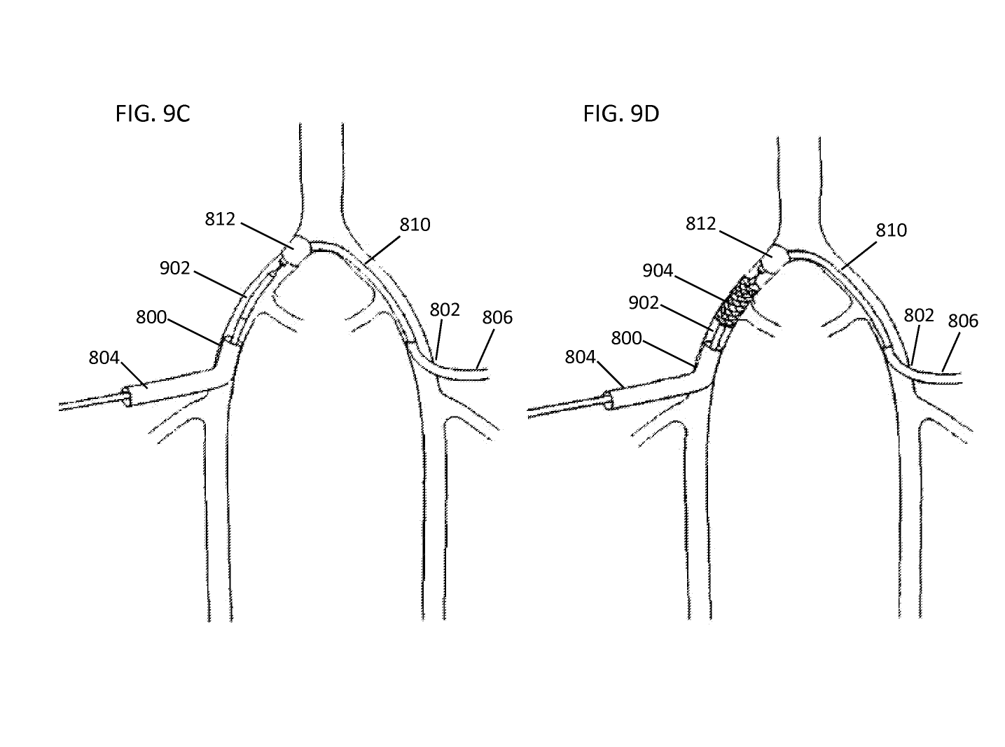

In instances where a vascular perforation is detected (e.g., during one of the bleed check as described above with respect to FIGS. 8A-8F or at some point during an endovascular procedure), the blood control system may be used to temporarily control bleeding through the vascular perforation. For example, FIGS. 9A-9D depict one variation of a method of controlling bleeding through a vascular perforation using the blood control system depicted in FIGS. 8A-8F. As shown there, EVP and BC access catheters ((804) and (806), respectively) may be positioned in the vasculature through EVP and BC access sites ((800) and (802), respectively), and the guidewire (808) may be positioned to form a track between the EVP and BC access sheaths, such as described above with respect to FIG. 8A. If a vascular perforation (900) is formed (e.g., from advancement or use of a EVP treatment device during performance of an endovascular procedure), any EVP treatment devices may be retracted from the ipsilateral vasculature, and the blood control catheter (810) may be advanced over the guidewire (808) to position the expandable member (812) of the blood control catheter (810) in the ipsilateral vasculature upstream of the vascular perforation (900), such as shown in FIG. 9A. The expandable member (812) may be expanded to stop blood flow past the expandable member (812), the IVS delivery catheter (816) may be advanced from the lumen of the blood control catheter (810), and the intra-vessel support (818) may be delivered from the IVS delivery catheter (816) to cover the vascular perforation (900), such as described above. With the intra-vessel support (818) covering the vascular perforation, the blood control system may temporarily control bleeding from the vascular perforation.

With the intra-vessel support (818) covering the vascular perforation (900), a covered-stent delivery device (902) may be introduced into the ipsilateral vasculature through the EVP access sheath (804), as shown in FIG. 9B. In variations where the guidewire (808) is positioned through the EVP access sheath (804), the covered-stent delivery device (902) may be advanced along the guidewire (808). In other instances, the covered-stent delivery device (902) may be advanced over a second guidewire (not shown) which may be advanced into the ipsilateral vasculature through the EVP access sheath (804).

With the stent delivery device (902) positioned between the EVP access sheath (804) and the blood control catheter (810), the intra-vessel support (818) may be recovered into the IVS delivery catheter (816) (e.g., using the retrieval mechanism (820)), such as shown in FIG. 9C. The covered-stent delivery device (902) may then be used to deliver a covered stent (904) to cover the vascular perforation (900), as shown in FIG. 9D. In some variations, radiopaque dye or other contrast agents may be introduced into the ipsilateral vasculature (e.g., through a lumen of the blood control catheter (810)) to assist in visualization of the vasculature and positioning the covered stent (904).

Once the covered stent (904) is deployed, the expandable member (812) of the blood control catheter (810) may be unexpanded, and a practitioner may check for subsequent bleeding. If no bleeding is detected, the blood control catheter (810) and the IVS delivery catheter (816) may be retracted into the contralateral vasculature, and the endovascular procedure may be resumed (e.g., one or more procedural catheters may be reintroduced into the vasculature via the EVP access sheath (804)). If the endovascular procedure has been completed, the blood control system may assist in closure of the EVP access site (800), such as described above with respect to FIGS. 8A-8F.

While the systems and methods described here are described with respect to the iliac vasculature, it should be appreciated that the blood control systems may be used to control bleeding in any suitable locations, such as subclavian arteries or other large vessels suitable for large bore access. Additionally, as mentioned above, the blood control systems described here may be used to close any suitable vascular perforation. For example, in some variations, the blood control system can be used to control bleeding for a vascular perforation in a trauma patient. Generally, a blood control catheter may be introduced into the vasculature via an access site and advanced to position an expandable member of the blood control catheter upstream of the vascular perforation. The expandable member may be expanded to block blood flow upstream of the perforation, and an IVS delivery catheter may deliver an intra-vessel support to temporarily cover the vascular perforation. A retrieval mechanism may maintain a connection between the intra-vessel support and the IVS delivery catheter, and the expandable member of the blood control catheter may be deflated to allow blood to flow past the expandable member, the retrieval mechanism, and through the intra-vessel support.

While the methods described above are generally described as using an intra-vessel support to at least temporarily cover an opening in a vessel wall, it should also be appreciated that the methods described above may be used to deploy an intra-vessel support to cover one or more vessels in which there is a risk of dissection or aneurysm rupture, which may temporarily provide support to that vessel. Additionally, in some variations, the systems and methods described here may be used to provide bleeding control during cerebral vascular procedures (e.g., procedures to treat cerebral aneurysms, AV malformations, AV fistulae, or the like), the blood control systems described here may be used to provide intravascular bleeding control while other therapeutic devices may be deployed to provide or effectuate the treatment.

We claim:

1. A blood control system comprising:
a blood control catheter, the blood control catheter comprising an expandable member on a distal end of the blood control catheter;
an intra-vessel support delivery catheter sized and configured to be slidably received in the blood control catheter; and
an intra-vessel support connected to the intra-vessel support delivery catheter by a retrieval mechanism,
wherein the intra-vessel support is moveable between an unexpanded configuration in which the intra-vessel support is positioned inside of the intra-vessel support delivery catheter and an expanded configuration in which the intra-vessel support is delivered from the intra-vessel support delivery catheter, and
wherein the retrieval mechanism is configured to retract the intra-vessel support from the expanded configuration to the unexpanded configuration.

2. The blood control system of claim 1 further comprising a guidewire.

3. The blood control system of claim 2 wherein the guidewire comprises a core wire and a flexible sheet attached to the core wire.

4. The blood control system of claim 3 wherein the guidewire further comprises an outer shaft, wherein the outer shaft is moveable between an advanced position to cover the flexible sheet and a retracted position to at least partially expose the flexible sheet.

5. The blood control system of claim 1 wherein the intra-vessel support comprises a stent graft.

6. The blood control system of claim 1 wherein the retrieval mechanism comprises a tether.
